(19) United States
(12) Patent Application Publication
(10) Pub. No.: US 2012/0059005 A1
(43) Pub. Date: Mar. 8, 2012

(54) COMBINATION OF (A) A PHOSPHOINOSITIDE 3-KINASE INHIBITOR AND (B) AN ANTIDIABETIC COMPOUND FOR USE IN THE TREATMENT OF PROLIFERATIVE DISEASES

(75) Inventors: Jose Baselga, Barcelona (ES); Serena Di Cosimo, Boville Ernica (IT); Carlos Garcia-Echeverria, Saint Cloud (FR); Wolfgang Hackl, Loerrach (DE); Sauveur-Michel Maira, Habsheim (FR); Russillo Michelangelo, Rome (IT); Violeta Serra Elizalde, Vilassar de Mar (ES)

(73) Assignee: NOVARTIS AG, BASEL (CH)

(21) Appl. No.: 13/319,536

(22) PCT Filed: May 12, 2010
(86) PCT No.: PCT/EP10/56538

§ 371 Date: Nov. 9, 2011

(30) Foreign Application Priority Data
May 15, 2009 (EP) 09160440.5

(51) Int. Cl. A61K 31/5377; A61K 31/437; A61P 35/00

(52) U.S. Cl. 514/235.8; 514/293

(57) ABSTRACT

The invention relates to a pharmaceutical combination which comprises (a) a phosphoinositide 3-kinase inhibitor compound and (b) an insulin sensitivity enhancer compound for the treatment of a proliferative disease, especially a solid tumor disease; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

### FIELD OF THE INVENTION

The invention relates to a pharmaceutical combination which comprises (a) a phosphoinositide 3-kinase (PI3k) inhibitor compound and (b) an antidiabetic compound and optionally at least one pharmaceutically acceptable carrier for simultaneous, separate or sequential use, in particular for the treatment of a proliferative disease, especially a proliferative disease in which the PI3k/Akt and/or RAS/MARK pathways are dysregulated; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

### BACKGROUND OF THE INVENTION

Several inhibitors of the PI3KJAKT/mTOR pathway are currently present in early clinical trials for the treatment of cancer. Some of them dramatically increase the blood glucose levels.

### SUMMARY OF THE INVENTION

The present invention relates to pharmaceutical combinations comprising (a) a phosphoinositide 3-kinase (MK) inhibitor compound and (b) an antidiabetic compound and optionally at least one pharmaceutically acceptable carrier for simultaneous, separate or sequential use., for the treatment of a proliferative disease, especially a proliferative disease in which the PI3k/Akt and/or RASIMAPK pathways are dysregulated.

The present invention also relates to pharmaceutical compositions comprising the combinations of (a) a phosphoinositide 3-kinase (Mk) inhibitor compound and (b) an antidiabetic compound and optionally at least one pharmaceutically acceptable carrier for simultaneous, separate or sequential use for the treatment of a proliferative disease, especially a proliferative disease in which the PI3K/Akt and/or RAS/MAPK pathways are dysregulated.

The present invention also relates to the use of such a combination for the preparation of a medicament for the treatment of a proliferative disease.

The present invention also relates to methods of treating a warm-blooded animal, especially a human, suffering from a proliferative disease in which the PI3K/Akt and/or RAS/MAPK pathways are dysregulated comprising administering a phosphoinositide 3-kinase (Mk) inhibitor compound and (b) an antidiabetic compound.

The present invention also relates to a commercial package or product comprising the combination as a combined preparation for simultaneous, separate or sequential use; and to a method of treatment of a warm-blooded animal, especially a human.

In one aspect of the invention the proliferative disease is a solid tumor, including breast cancer, ovarian cancer, cancer of the colon such as e.g, colorectal cancer (CRC), and generally the GI (gastro-intestinal) tract, cervix cancer, lung cancer such as e.g. non-small-cell lung cancer (NSCLC), head and neck cancer, bladder cancer, kidney cancer such as e,g, renal cell carcinoma (RCC), liver cancer, brain cancer, endometrial cancer, neuroendocrine tumors, thyroid cancer, pancreatic cancer, cancer of the prostate or Kaposi's sarcoma.

In another aspect of the invention, the proliferative disease Peutz-Jeghers syndrome, which is characterized by intestinal hamartomas and increased incidence of epithelial cancers,

### DETAILED DESCRIPTION OF THE FIGURES

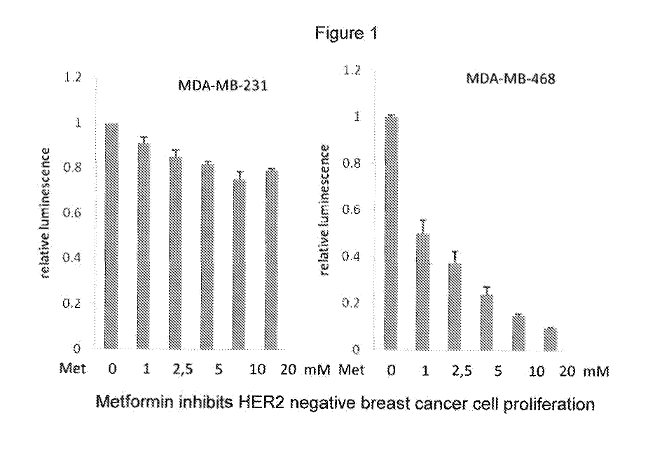

FIG. 1 illustrates metformin as an inhbitor of HER2 negative breast cancer cell proliferation

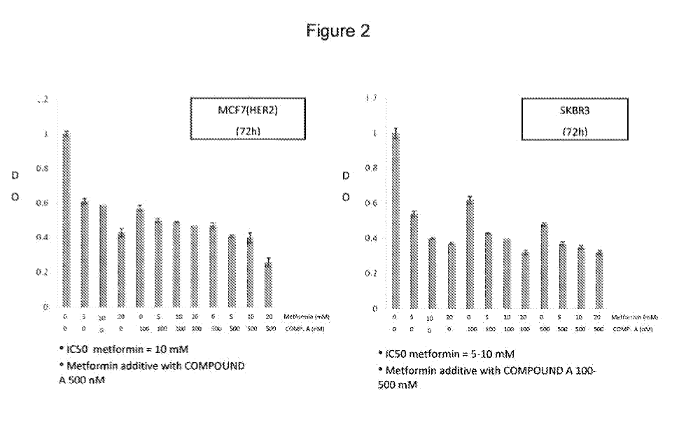

FIG. 2 illustrates the combined treatment of COMPOUND A plus metformin results in an inhibitory effect on cell proliferation

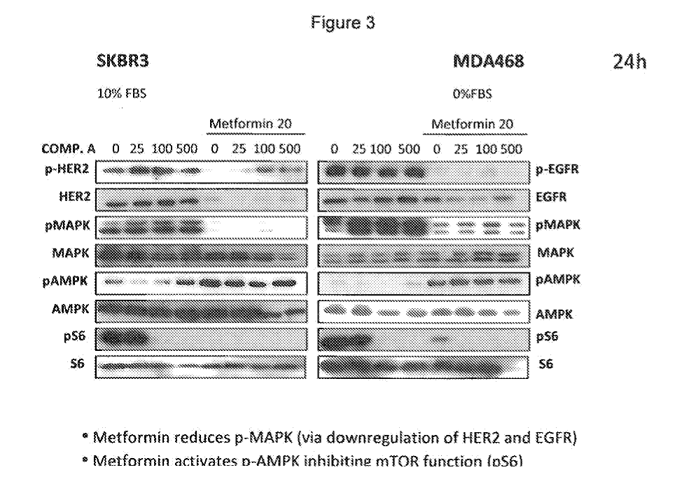

FIG. 3 illustrates the biochemical effects of metformin and COMPOUND A where metformin reduces p-MAPK (via downregulation of HER2 and EGFR) and metformin activates p-AMPK inhibiting mTOR function (pS6).

### DETAILED DESCRIPTION OF THE INVENTION

WO2006/122806 describes imidazoquinoline derivatives, which have been described to inhibit the activity of lipid kinases, such as PI3-kinases. Specific imidazoquinoline derivatives which are suitable for the present invention, their preparation and suitable pharmaceutical formulations containing the same are described in WO2006/122806 and include compounds of formula

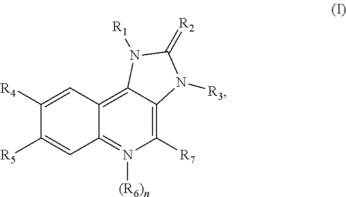

wherein

R₁ is naphthyl or phenyl wherein said phenyl is substituted by one or two substituents independently selected from the group consisting of Halogen, lower alkyl unsubstituted or substituted by halogen, cyano, imidazolyl or triazolyl; cycloalkyl: amino substituted by one or two substituents independently selected from the group consisting of lower alkyl, lower alkyl sulfonyl, lower alkoxy and lower alkoxy lower alkylamino: piperazinyl unsubstituted or substituted by one or two substituents independently selected from the group consisting of lower alkyl and lower alkyl sulfonyl, 2-oxo-pyrrolidinyl; lower alkoxy lower alkyl; imidazolyl; pyrazolyl; and triazolyl;

R₂ is O or S;

R₃ is lower alkyl;

R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy

R₅ is hydrogen or halogen;

n is 0 or 1;

R₆ is oxido;

with the proviso that if n=1, the N-atom bearing the radical R₆ has a positive charge;

R₇ is hydrogen or amino;

or a tautomer thereof, or a pharmaceutically acceptable salt, or a hydrate or solvate thereof.

The radicals and symbols as used in the definition of a compound of formula I have the meanings as disclosed in WO2006/122806 which publication is hereby incorporated into the present application by reference,

A preferred compound of the present invention is a compound which is specifically described in WO2006/122806, A very preferred compound of the present invention is 2-methyl-2[4-(3-methyl-2-oxo-8-quinolin-3-0-2,3-dihydro-imidazo[4,5-c]quinolin-1-yl)-phenyl]-propioriitrile and its manotosylate salt (COMPOUND A), The synthesis of 2-methyl-2[4-(3-methyl-2-oxo-8-quinolin-3-yl-2,3-dihydro-imidazo[4,5-c]quinolin-1-yl)-phenyl]-propionitrile is for instance described in WO2006/122806 as Example 1. Another very preferred compound of the present invention is 8-(6-methoxy-pyridin-3-yl)-3-methyl-1-(4-piperazin-1-yl-3-trifluoromethyl-phenyl)-1,3-dihydro-imidazo [4,5-c]quinolin-2-one (COMPOUND B), The synthesis of 8-(6-methoxy-pyridin-3-yl)-3-methyl-1-(4-piperazin-1-yl-3-trifluorornethyl-phenyl)-1, 3-dihydro-imidazo[4,5-c]quinolin-2-one is for instance described in WO2006/122806 as Example 86.

WO07/084786 describes pyrirnidine derivatives, which have been found the activity of lipid kinases, such as PI3-kinases. Specific pyrimidine derivatives which are suitable for the present invention, their preparation and suitable pharmaceutical formulations containing the same are described in WO07/084786 and include compounds of formula II.

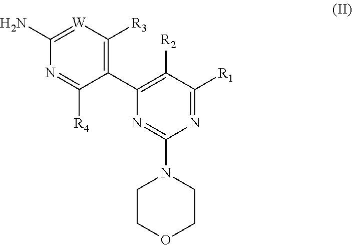

or a stereoisomer, tautomer, or pharmaceutically acceptable salt thereof, wherein,
W is CR_{w} or N, wherein R_{w} is selected from the group consisting of
(1) hydrogen,
(2) cyano,
(3) halogen,
(4) methyl,
(5) trifiuoromethyl,
(6) sulfonamido;
R₁ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyt,
(8) substituted and unsubstituted aryl,
(9) substituted and unsubstituted heteroaly,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(22) —SO₂R₁ₐ, and
(23) —SO₂NR₁ₐR_{1b},
wherein R₁ₐ, and R_{1b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
R₂ is selected from the group consisting
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b},
wherein R₂ₐ, and R_{2b} are independently selected from the group consisting of
(a) hydrogen, and
(b) substituted or unsubstituted alkyL
R3 is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(13) —NR₃ₐR_{3b},
(14) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, and
(20) —SO₂NR₃ₐR_{3b},
wherein R₃ₐ, and R_{3b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and
R₄ is selected from the group consisting of
(1) hydrogen, and
(2) halogen.

The radicals and symbols as used in the definition of a compound of formula have the meanings as disclosed in WO07/084786 which publication is hereby incorporated into the present application by reference.

A preferred compound of the present invention is a compound which is specifically described in WO07/084786. A very preferred compound of the present invention is 5-(2,6-di-morpholin-4-yl-pyrimidin-4-yl)-4-trifluoromethyl-pyridin-2-ylamine and its hydrochloride salt (COMPOUND C). The synthesis of 5-(2,6-di-morpholin-4-yl-pyrimidin-4-yl)-4-trifluoromethyl-pyridin-2-ylamine is described in WO077084786 as Example 10.

In one aspect, the present invention pertains to a combination such as a combined preparation or a pharmaceutical composition which comprises (a) a phosphoinositide 3-kinase (MK) inhibitor compound and (b) a antidiabetic which is an insulin sensitizer and is an activator of AMP-activated protein kinase (AMPK), such as e,g, a biguanide or a thiazolidinedione (glitazone).

Exemplary biguanide compounds include drugs that are insulin sensitivity enhancers and e.g. useful in controlling or managing non-insulin-dependent diabetes mellitus (NIDDM). Non-limiting examples of biguanides include metformin, phenformin or buformin and the like and pharmaceutically acceptable salts, or isomers thereof. In a preferred embodiment, the biguanide is metformin. The preparation of metformin (dimethyldiguanide) and its hydrochloride salt is state of the art and was disclosed first by Emil A. Werner and James Bell, J. Chem. Soc. 121, 1922, 1790-1794. Metformin, can be administered e.g. in the form as marketed under the trademarks GLUCOPHAGE™.

In another aspect, the present invention relates to a combination such as a combined preparation or a pharmaceutical composition which comprises (a) a phosphoinositide 3-kinase (PI3K) inhibitor compound and (b) metformin.

In another embodiment the antidiabetic is a thiazolidinedione (glitazone). Exemplary glitazones include 5-{[4-(2-(5-ethyl-2-pyridyl)ethoxy)phenyl]-methyl}thiazolidine-2, 4-dione (pioglitazone, EP 0 193 256 A1), 5-{[4-(2-(methyl-2-pyridinyl-amino)-ethoxy) phenyl]methyl}thiazolidine-2,4-dione (rosiglitazone, EP 0 306 228 A1), 5-{[4-((3,4-dihydro-6-hydroxy-2,5,7,8-tetramethyl-2H-1-benzopyran-2-yl) methoxy)-phenyl]-methyl}thiazolidine-2,4-dione (troglitazone, EP 0 139 421), (S)-((3, 4-dihydro-2-(phenyl-methyl)-2H-1-benzopyran-6-yl)methyl-thiazolidine-2,4-dione (englitazone, EP 0 207 605 B1), 5-(2,4-dioxothiazolidin-5-ylmethyl)-2-methoxy-N-(4-trifluoromethyl-benzyl) benzamide (KRP297, JP 10087641-A), 5-[6-(2-fluoro-benzyloxy)naphthalen-2-ylmethyl]thiazolidine-2,4-dione (MCC555, EP 0 604 983 B1), 5-{[4-(3-(5-methyl-2-phenyl-4-oxazolyl)-1-oxopropyl)-phenyl]-methyl}-thiazolidine-2, 4-dione (darglitazone, EP 0 332 332), 5-(2-naphthylsulfonyl)-thiazolidine-2,4-dione (AY-31637, U.S. Pat. No. 4,997,948), 5-{[4-(1-methyl-cyclohexyl)methoxy)-phenyl]methyl}-thiazolidine-2, 4-dione (ciglitazone, U.S. Pat. No. 4,287,200), 5-{4-[(6-methoxy-1-methyl-1H-benzimidazol-2-yl) methoxy]benzyl}-1,3-thiazolidine-2,4-dione (rivoglitazone, CAS-No. 185428-18-6) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The preparation of DRF2189 and of 5-{([4-(2-(2,3-dihydroindol-1-yl)ethoxy)phenyl]methyl}-thiazolidine-2, 4-dione is described in B,B. Lohray et al,, J. Med, Chem. 1998, 41, 1619-1630; Examples 2d and 3g on pages 1627 and 1628. The preparation of 5-[3-(4-chlorophenyl])-2-propynyl]-5-phenylsulfonyl)thiazolidine-2,4-dione and the other compounds in which A is phenylethynyl mentioned herein can be carried out according to the methods described in J. Wrobel at al., J. Med. Chem. 1998, 41, 1084-1091,

In particular, MCC555 can be formulated as disclosed on page 49, lines 30 to 45, of EP 0 604 983 81; englitazone as disclosed from page 6, line 52, to page 7, line 6, or analogous to Examples 27 or 28 on page 24 of EP 0 207 605 81; and dairglitazone and 5-{4-[2-(5-methyl-2-phenyl-4-oxazolyl)-ethoxy)]benzyl}-thiazolidine-2,4-dione (BM-13.1246) can be formulated as disclosed on page 8, line 42 to line 54 of EP 0 332 332 81. AY-31637 can be administered as disclosed in column 4, lines 32 to 51 of U.S. Pat. No. 4,997,948 and rosiglitazone as disclosed on page 9, lines 32 to 40 of EP 0 306 228 A1, the latter preferably as its maleate salt. Rosiglitazone can be administered in the form as it is marketed e.g. under the trademark AVANDIA™. Troglitazone can be administered in the form as it is marketed e.g. under the trademarks ReZulih™, PRELAY™, ROIVIOZIN™ (in the United Kingdom) or NOSCAL™ (in Japan). Pioglitazone can be administered as disclosed in Example 2 of EP 0 193 256 A1, preferably in the form of the monohydrochloride salt. Corresponding to the needs of the single patient it can be possible to administer pioglitazone in the form as it is marketed e.g. under the trademark ACTOS™. Ciglitazone can, for example, be formulated as disclosed in Example 13 of U.S. Pat. No. 4,287,200. Other activators of AMP-activated protein kinase (AMPK) that are useful for the present invention include compounds described and cited in Zhou at al, Acta Physiologica 2009, 196, 175-190, including the compounds described in WO 2008/016278, US 2005/0038068, WO 2007/062568, WO 2008/006432, WO 2008/083124, WO 2007/005785, FR2846656, EP 1 754 483 A1, WO 2006/071095 A1, which are herewith incorporated by reference.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single.

In one embodiment of the invention, (a) the phosphoinositide 3-kinase (PI3K) inhibitor compound inhibitor is COMPOUND A, COMPOUND B or COMPOUND C.

The term “treating” or “treatment” as used herein comprises a treatment effecting a delay of progression of a disease. The term “delay of progression” as used herein means administration of the combination to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

In one embodiment of the present invention, the proliferative disease is a solid tumor. The term “solid tumor” especially means breast cancer, ovarian cancer, cancer of the colon such as e.g. colorectal cancer (CRC), and generally the GI (gastro-intestinal) tract, cervix cancer, lung cancer such as e.g. non-small-cell lung cancer (NSCLC), head and neck cancer, bladder cancer, kidney cancer such as e.g. renal cell carcinoma (RCC), liver cancer, brain cancer, endometrial cancer, neuroendocrine tumors, thyroid cancer, pancreatic cancer, cancer of the prostate or Kaposi's sarcoma.

In a preferred embodiment, the proliferative disease is lung cancer,in particular lung tumors carrying a germline mutations in serine/threonine kinase 11 (STK11, also called LKB1). Inactivating somatic mutations of LBK1 have been reported in primary human lung adenocarcinomas. Thus, germline mutations in LKBI have been found in 34% and 19% of 144 analysed human lung adenocarcinomas and squarnos cell carcinomas, respectively. A loss-of-function mutation of LKB1 may also strongly cooperate with a dysfunctional activation of the MK and/or RAS/MAPK pathways, which are also common alterations in lung tumors. It has now been found that lung tumors carrying a loss-of-function mutation of LKB1 can be effectively treated with the COMBINATION OF THE INVENTION.

In a preferred embodiment, the proliferative disease Peutz-Jeghers syndrome, which is characterized by intestinal hamartomas and increased incidence of epithelial cancers.

Proliferative diseases that may be treated with the COMBINATION OF THE INVENTION in accordance with another embodiment of the present invention, include Breast Cancer, Ovarian Cancer, Colon Cancer, Pancreas Cancer, Melanoma, Head and Neck Cancer, Endometrial Cancer and Brain Cancer.

The present combination inhibits the growth of solid tumors, but also liquid tumors. Furthermore, -depending on the tumor type and the particular combination used a decrease of the tumor volume can be obtained. The combinations disclosed herein are also suited to prevent the metastatic spread of tumors and the growth or development of rnicrometastases. The combinations disclosed herein are in particular suitable for the treatment of poor prognosis patients, especially such poor prognosis patients having lung tumors.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

It will be understood that references to the combination partners (a) and (b) are meant to also include the pharmaceutically acceptable salts. If these combination partners (a) and (b) have, for example, at least one basic center, they can form acid addition salts. Corresponding acid addition salts can also be formed having, if desired, an additionally present basic center. The combination partners (a) and (b) having an acid group (for example COOH) can also form salts with bases. The combination partner (a) or (b) or a pharmaceutically acceptable salt thereof may also be used in form of a hydrate or include other solvents used for crystallization,

A combination which comprises (a) a phosphoinositide 3-kinase inhibitor compound and (b) a biguanide insulin sensitivity enhancer, in which the active ingredients are present in each case in free form or in the form of a pharmaceutically acceptable salt and optionally at least one pharmaceutically acceptable carrier, will be referred to hereinafter as a COMBINATION OF THE INVENTION,

The COMBINATION OF THE INVENTION are both synergistic and additive advantages, both for efficacy and safety. Therapeutic effects of combinations of a phosphoinositide 3-kinase inhibitor compound with a compound which modulates the biguanide insulin sensitivity enhancer can result in lower safe dosages ranges of each component in the combination. Moreover, an insulin sensitivity enhancer is useful in overcoming the potential increase in blood glucose caused by modulators of PI3k signaling,

The pharmacological activity of a COMBINATION OF THE INVENTION may, for example, be demonstrated in a clinical study or in a test procedure as essentially described hereinafter. Suitable clinical studies are, for example, open label non-randomized, dose escalation studies in patients with advanced solid tumors. Such studies can prove the additive or synergism of the active ingredients of the COMBINATIONS OF THE INVENTION. The beneficial effects on proliferative diseases and/or glucose homeostasis can be determined directly through the results of these studies or by changes in the study design which are known as such to a person skilled in the art. Such studies are, in particular, suitable to compare the effects of a monotherapy using the active ingredients and a COMBINATION OF THE INVENTION. Preferably, the combination partner (a) is administered with a fixed dose and the dose of the combination partner (b) is escalated until the Maximum Tolerated Dosage is reached.

It is one objective of this invention to provide a pharmaceutical composition comprising a quantity, which is therapeutically effective against a proliferative disease comprising the COMBINATION OF THE INVENTION, In this composition, the combination partners (a) and (b) can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including man. Alternatively, when the agents are administered separately, one can be an enteral formulation and the other can be administered parenterally.

The novel pharmaceutical composition contain, for example, from about 10% to about 100%, preferably from about 20% to about 80%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, for example, water, glycols, oils, alcohols, flavoring agents, preservatives, coloring agents or carriers such as starches, sugars, microcristalline cellulose, diluents, granulating agents, lubricants, binders, disintegrating agents and the like in the case of oral solid preparations such as for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

In particular, a therapeutically effective amount of each of the combination partner of the COMBINATION OF THE INVENTION may be administered simultaneously or sequentially and in any order, and the components may be administered separately or as a fixed combination. For example, the method of delay of progression or treatment of a proliferative disease according to the invention may comprise (i) administration of the first combination partner in free or pharmaceutically acceptable salt form and (ii) administration of the second combination partner in free or pharmaceutically acceptable salt form, simultaneously or sequentially in any order, in jointly therapeutically effective amounts, preferably in synergistically effective amounts. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The COMBINATION OF THE INVENTION can be a combined preparation or a pharmaceutical composition.

Moreover, the present invention relates to a method of treating a warm-blooded animal having a proliferative disease comprising administering to the animal a COMBINATION OF THE INVENTION in a quantity which is therapeutically effective against said proliferative disease.

Furthermore, the present invention pertains to the use of a COMBINATION OF THE INVENTION for the treatment of a proliferative disease and for the preparation of a medicament for the treatment of a proliferative disease.

Moreover, the present invention provides a commercial package comprising as active ingredients COMBINATION OF THE INVENTION, together with instructions for simultaneous, separate or sequential use thereof in the delay of progression or treatment of a proliferative disease.

Preferred embodiments of the invention are represented by combinations comprising
COMPOUND A, COMPOUND B or COMPOUND C and metformin,
COMPOUND A, COMPOUND B or COMPOUND C and phenformin,
COMPOUND A, COMPOUND B or COMPOUND C and pioglitazone,
COMPOUND A, COMPOUND B or COMPOUND C and rivoglitazone,
COMPOUND A, COMPOUND B or COMPOUND C and rosiglitazone
COMPOUND A, COMPOUND B or COMPOUND C and ciglitazone
COMPOUND A, COMPOUND B or COMPOUND C and darglitazone
COMPOUND A, COMPOUND B or COMPOUND C and englitazone.

In further aspects, the present inventions provides
a combination which comprises (a) a COMBINATION OF THE INVENTION, wherein the active ingredients are present in each case in free form or in the form of a pharmaceutically acceptable salt or any hydrate thereof, and optionally at least one pharmaceutically acceptable carrier; for simultaneous, separate or sequential use;
a pharmaceutical composition comprising a quantity which is jointly therapeutically effective against a proliferative disease of a COMBINATION OF THE INVENTION and at least one pharmaceutically acceptable carrier;
the use of a COMBINATION OF THE INVENTION for the treatment of a proliferative disease;
the use of a COMBINATION OF THE INVENTION for the preparation of a medicament for the treatment of a proliferative disease;
the use of a combination COMBINATION OF THE INVENTION wherein the PI3K inhibitor is selected from COMPOUND A, COMPOUND B or COMPOUND C: and
the use of a COMBINATION OF THE INVENTION wherein the biguanide insulin sensitivity enhancer compound is a biguanide, e.g. metformin or phenformin;
the use of a COMBINATION OF THE INVENTION wherein the biguanide insulin sensitivity enhancer compound is a glitazone, e.g. pioglitazone, rivoglitazone, rosiglitazone, ciglitazone, darglitazone, englitazone.

Moreover, in particular, the present invention relates to a combined preparation, which comprises (a) one or more unit dosage forms of a phosphoinositide 3-kinase inhibitor compound and (b) a biguanide insulin sensitivity enhancer compound.

Furthermore, in particular, the present invention pertains to the use of a combination comprising (a) a phosphoinositide 3-kinase inhibitor compound and (b) a biguanide insulin sensitivity enhancer compound for the preparation of a medicament for the treatment of a proliferative disease and/or overcoming the potential increase in blood glucose caused by inhibition of the PI3K/Akt pathway.

The effective dosage of each of the combination partners employed in the COMBINATION OF THE INVENTION may vary depending on the particular compound or pharmaceutical composition employed, the mode of administration, the condition being treated, the severity of the condition being treated, Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics f the active ingredients' availability to target sites.

When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

COMPOUND A may be administered to a human in a dosage range varying from about 25 to 1600 mg /day.

COMPOUND B may be administered to a human in a dosage range varying from about 2.5-150 mg/3×/week or 2.5 to 75 mg/day.

COMPOUND C may be administered to a human in a dosage range varying from about 12.5 to 600 mg/day.

Metformin may be administered to a human e.g. 850 mg bid.

The beneficial effects of the COMBINATION OF THE INVENTION can also be determined by other test models known as such to the person skilled in the pertinent art.

The following examples are offered by way of illustration and are not intended to limit the scope of the invention. Variations, modification and other implementations of what is described herein will occur to those of ordinary skill in the art without departing from the spirit and the essential characteristics of the present teachings. The cell lines mentioned therein are not thougt to limit the scope of the invention as they are merely representatives and may be replaced with different cell lines and tumor cells for which they are representatives. Accordingly the scope of the invention is to be defined not by the preceding illustrative description but instead by the following claims, and all changes that come within the meaning and range of equivalency of the claims are intended to be embraced therein.

### Example 1

### Dual-Targeting of AMPK and PI3K/mTOR in a Panel of Breast Cancer Cell Lines

MCF-7(HER2), SK-BR-3, MDA-MB-231 and MDA-MB-468 breast cancer cells are treated with different doses of 2-Methyl-2-[4-(3-methyl-2-oxo-8-quinolin-3-yl-2, 3-dihydro-imidazo[4,5-c]quinolin-1-yl)-phenyl]-propionitrile, also known as COMPOUND A, metformin or both agents in combination. Levels of phosphorylated and total AMPK, MAPK, EGFR, HER and S6 ribosomal protein are evaluated by western blot. Cell proliferation analyses are performed in triplicates using the WST-1 and crystal violet colorimetric assays.

Metformin induces dose-dependent growth inhibition of MCF-7(HER2), SK-BR-3, MDA-MB-231 and MDA-MB-468 breast cancer cell lines as illustrated in FIGS. 1 and 2. The combined treatment of COMPOUND A plus metformin results in an inhibitory effect on cell proliferation greater than with either treatment alone as illustrated in FIG. 2. Metformin activates AMPK reducing mTORC1 activity and decreasing the levels of p-S6 ribosomal protein, Treatment with metformin is also associated with reduced receptor tyrosine kinase (EGFR and HER2) expression and decreased p-MAPK. COMPOUND A potently decreases p-AKT and p-S6. However, as described for other mTOR inhibitors, COMPOUND A increases MAPK phosphorylation by transactivation of several receptors tyrosine kinase (RT s) including EGFR and HER2. Metformin counteracts the MARK pathway transactivation induced by COMPOUND A likely by downregulating EGFR and/or HER2 as illustrated in FIG. 3. This data provides the rationale of combining metformin with PI3/mTOR inhibitors in EGFR or HER2 over-expressing cells.

The combination of metformin and COMPOUND A inhibits the growth of EGFR positive and HER2 positive breast cancer cell lines. We provide the rationale for targeting both AMPK and PI3K/Akt/mTOR pathways to elicit strong anti-tumor effects in breast cancer.

### Example 2

### Combination Effect of PI3K Inhibitors and Metformin on A549 Non-Small Cell Lung Tumors in Nude Mice Xenograft Model

A549 human non-small cell lung cancer (NSCLC) cells are treated with different doses of the 2-Methyl-2-[4-(3-methyl-2-oxo-8-quinolin-3-yl-2,3-dihydro-imidazo[4, 5-c]quinolin-1-yl)-phenyl]-propionitrile monotosylate salt (also known as COMPOUND A) or 5-(2,6-di-morpholin-4-yl-pyrimidin-4-yl)-4-trifluoromethyl-pyridin-2-ylamine hydrochloride salt (also known as COMPOUND C), a single dose of metformin or both agents in combination. The A549 human NSCLC cells (ATCC-CCL-185, available from American Type Culture Collection, Rockville, Md. US) display characteristics of type II alveolar epithelial cells (Lieber et al, Int, J, Cancer 17(1): 62-70 [1976]), The A549 NSCLC cells are homozygous for mutations in the tumor suppressor genes, CDKN2A and STK11 (also called LKB1), and in KRAS. (Wellcome Trust Sanger Institute, Catalogue of Somatic Mutations in Cancer, Cosmic ID No, 905949, available at website http://www.sanger.ac.uk/peri/genetics/CGP/core_line_viewer?action=sample&name=A549.)

A549 tumor cells are grown in Kaighn's modified Ham's F12 medium containing 100 units/mL penicillin G sodium, 100 μg/mL streptomycin sulfate, 25 μg/mL gentamicin, 100% fetal bovine serum, 2 mM glutamine, and 1 mM sodium pyruvate. The cells are cultured in tissue culture flasks in a humidified incubator at 37° C., in an atmosphere of 5% CO₂ and 95% air. The cells are harvested for injection into 9-week old female nu/nu (nude) mice (Harlan Laboratories, Indianapolis, Ind.) by detaching the monolayers with 2X trypsin and resuspending at 5×10⁷cells/mL in cold phosphate-buffered saline containing 50% Matrigel.

0.2 mL of A549 cell suspension (1×10⁷ cells) is injected subcutaneously in the right flank of 9-week old female flu/flu (nude) mice (Harlan Laboratories, Indianapolis, Ind.) having a body weight (BW) range of 19.9-27.3 g on Day 1 of the study, Tumors are callipered in two dimensions to monitor their growth as their mean volume approached 150-220 mm³, Twenty-two days after implantation, the mice are sorted into 11 groups of eight or nine mice having individual tumor sizes of 108-221 mm³. Tumor volume in mm³ is determined using the formula [(width)²×(length)]12, where width width of the tumor in mm and length=length of the tumor in mm. Tumor weight can be estimated with the assumption that 1 mg is equivalent to 1 mm³ of tumor volume.

The 11 groups of nude mice are treated as follows. One group of nine mice serves as Controls (C or Control) for all analyses and is administered intraperitoneally (i,p,) 50 mM sodium acetate at pH 4 (Vehicle 1) and then administered by oral gavage (p.o.) a composition comprising 10% N-methylpyrrolidone: 90% polyethylene glycol 300 (PEG300) (Vehicle 2). All treatments with metformin (metformin hydrochloride, Glucophage®, Bristol-Myers Squibb Company) monotherapy are administered intraperitoneally (i.p.) once daily at a single dose of 192.3 mg/kg metformin until the end of the study as provided in the Results Table 1. Metformin is dissolved in 50 mM sodium acetate at pH 4 for dosing,

AH treatments with the Compound A or Compound C monotherapy are administered at varying doses by oral gavage (p.o.) once daily until the end of the study as provided in the Results Table 1. Compound A and Compound C are stored at −20° C. protected from light. Stock solutions (10X) in 100% N-methylpyrrolidone (NMP) are prepared every five days, aliquotted, and stored in the dark at room temperature. On each treatment day, stock solution aliquots are diluted with polyethylene glycol (PEG300) to provide the formulated drug (Compound A or Compound C) in 10% NMP: 90% PEG300. Dosing solutions are protected from light, and the formulated drug is administered within 1 hour after preparation.

For combination therapies, Compound A or Compound C are administered by oral gavage (p.o.) within 30 minutes after the intraperitoneal (i.p.) administration of metformin , except on Day 20 when Compound A or Compound C is given immediately after metformin. Compound A, Compound C and metformin are prepared and administered as disclosed above for the monotherapy and in Results Table 1.

Paclitaxel (Natural Pharmaceuticals, Inc., Beverly, Mass., USA) is administered by bolus tail-vein injections (i v) once daily on alternate days for five doses. Paclitaxel is dissolved in 50% ethanol and 50% Cremophor® EL to prepare a 10X stock solution stored at room temperature. On each day of dosing, an aliquot of the paclitaxel stock solution is diluted with 5% dextrose in water to yield a dosing solution containing 5% ethanol and 5% Cremophor®EL.

In all groups, the dosing volume of 10 mL/kg (0.2 mL/ 20 g mouse) is scaled to the weight of each animal as determined on the day of dosing, except on weekends when the previous BW is carried forward. Acceptable toxicity for the maximum tolerated dose (MTD) is defined as a group mean BW loss of less than 15% during the test, and not more than 10% treattnent-related mortality. Any animal with BW losses exceeding 15% for three consecutive measurements, or with a BW loss exceeding 20% for one measurement, is designated to be euthanized.

Short-term efficacy for tumor growth inhibition in A549 cells is determined on Day 20, the day on which the Control mean tumor volume nearly attained the 500 m³ endpoint. By Day 20, no tumors had progressed to the endpoint; but 6 animals had died prior to Day 20. Statistical and graphical analyses was conducted by determining the difference in tumor volume between Day 1 (the start of dosing) and the endpoint day for each animal that remained on study on Day 20. Antitumor activity is expressed as % T/C (comparing the mean tumor volume change between the endpoint day and Day 1 for the treatment group to the Control), or % T/T₀ (comparing the mean tumor volume change between the endpoint day and Day 1 for the treatment group to its tumor volume at the beginning of the experiment (T₀), A T/C≦40% generally indicates potential therapeutic activity. A partial regression indicates that the tumor volume was 50% or less of its initial volume on Day 1 for three consecutive measurements during the study, and equal to or greater than 13.5 mm³ for one or more of these three measurements. A complete regression indicates that the tumor volume was less than 13.5 mm³ for three consecutive measurements during the course of the study.

Results Table 1 summarizes results for A549 tumors, for the standard 20 day experiment. The metformin monotherapy at 192.3 mg/kg did not appear active in the A549 human NSCLC xenograft model in a 20-day tumor growth inhibition assay, The response to paclitaxel is consistent with prior results reported in this xenograft model.

The combined treatment of 32.7 mg/kg of COMPOUND C plus metformin results in −17% T/T₀ and significant median tumor reduction (p<0.01) as compared to the Control but improves non-significantly as compared to the COMPOUND C monotherapy. Combined treatment of 32.7 mg/kg of COMPOUND C plus metformin further results in significant median tumor reduction (p<0.001) as compared to the metformin monotherapy. Combination therapy with 54.4 COMPOUND C and metformin was terminated early due to toxicity.

The combined treatment of 41.1 mg/kg of COMPOUND A plus mefformin results in −27% T/T₀ with significant tumor reduction (p<0.001) as compared to Control but improves non-significantly as compared to the COMPOUND A monotherapy, Combined treatment of 41.1 mg/kg of COMPOUND A plus metformin further results in significant median tumor reduction. Combination therapy with 68.5 mg/kg of COMPOUND A plus metformin resulted in a −30% T/T₀ as compared to Control. This combination was not evaluable after one death among the group of eight mice exceeded 10% mortality limitations.

The combination treatment of COMPOUND A plus metformin and COMPOUND C plus metformin inhibits the growth of human NSCLC cell lines. It is demonstrated that the combination treatment of COMPOUND A plus metformin and COMPOUND C plus metformin in improved growth inhibition of human NSCLC cell lines as compared to Control and/or metformin monotherapy.

TABLE 1
Antitumor effect of PI3K inhibitors and metformin, alone and in
combination, on A549 non-small cell lung tumors in nude mice

| | | | Mean Tumor | | | | |
|---|---|---|---|---|---|---|---|
| | Dose | Route, | Vol. Change | | | Mean Body | Dead/ |
| Compound | (mg/kg) | Schedule | (mm³) | Regression | T/C or T/T₀ | Weight Change | Total |
| Vehicle 1/ | — | i.p., 1x daily | 318 | None | — | — | 0/9 |
| Vehicle 2 | | p.o., 1x daily | | | | | |
| Metformin | 192.3 | i.p., 1x daily | 221 | None | 69% (ns) | — | 1/9 |
| Compound C | 32.7 | p.o., 1x daily | 63 | None | 20% (ns) | −0.8%, Day 2 | 0/8 |
| Compound C | 54.4 | p.o., 1x daily | −80 | None | −48% (T/T₀, | −3.6%, Day 5 | 0/9 |
| | | | | | p < 0.001) | | |
| Compound A | 41.1 | p.o., 1x daily | 58 | None | 18% (ns) | — | 0/8 |
| Compound A | 68.5 | p.o., 1x daily | −51 | 3 Partial | −31% (T/T₀, | −0.8%, Day 4 | 0/9 |
| | | | | | p < 0.001) | | |
| Metformin | 192.3 | i.p., 1x daily | −27 | None | −17% (T/T₀, | — | 0/8 |
| Compound C^{†} | 32.7 | p.o., 1x daily | | | p < 0.01) | | |
| Metformin | 192.3 | i.p., 1x daily | 80 | None | 25% (ne) | −4%, Day 5 | 2/9 |
| | | until day 7^{‡} | | | | | |
| Compound C | 54.4 | p.o., 1x daily | | | | | |
| | | until day 7^{‡} | | | | | |
| Metformin | 192.3 | i.p., 1x daily | −46 | 1 Partial | −27% (T/T₀, | — | 1/8 |
| Compound A | 41.1 | p.o., 1x daily | | | p < 0.001) | | |
| Metformin | 192.3 | i.p., 1x daily^{††} | −48 | None | −30 (T/T₀, | −4.4%, Day 9 | 2/9 |
| Compound A | 68.5 | p.o., 1x daily | | | ne) | | |
| Paclitaxel | 30 | i.v., 1x on | 9 | None | 3% | −14%, Day 9 | 0/8 |
| | | alternate days | | | (p < 0.05) | | |
| | | for 5 doses | | | | | |

^{†}Animals # 5, 6, 7 and 8 received 68.5 mg/kg Compound A on Day 17 instead of 32.7 mg of Compound C
^{‡}Animals # 7, 8 and 9 received metformin p.o. instead of i.p. on Day 2.
^{††}Animals #7, 8 and 9 received metformin p.o. instead of i.p. on Day 2.
T/C = 100 × (ΔT/ΔC) = percent change between Day 1 and D 20 in the mean tumor volume of a treated group (ΔT) compared with change in Control (ΔC).
T/T₀ = 100 × (ΔT/ T₀) = percent change between Day 1 and D 20 in the mean tumor volume of a treated group (ΔT) compared with its initial volume, when ΔT < 0.
Statistical Significance (Kruskal-Wallis with post hoc Dunn's multiple comparison test) vs. indicated group of Control or T₀: ne = not evaluable; ns = not significant at p > 0.05
Mean BW Change = lowest group mean body weight, as change from Day 1 up to Day 20, “—” indicates no decrease in mean body weight was observed.

### Example 3

### Combination Effect of PI3K Inhibitors and Metformin on H520 Non-Small Cell Lung Tumors in Nude Mice Xenograft Model

EGFR-null H520 human non-small cell lung cancer (NSCLC) cells are treated with different doses of 2-Methyl-2-[4-(3-methyl-2-oxo-8-quinolin-3-yl-2, 3-dihydro-imidazo[4,5-c]quinolin-1-yl)-phenyl]-propionitrile monotosylate salt (also known as COMPOUND A) or 5-(2,6-di-morpholin-4-yl-pyrimidin-4-yl)-4-trifluoromethyl-pyridin-2-ylamine hydrochloride salt (also known as COMPOUND C), a single dose of metformin or both agents in combination, The H520 human NSCLC cells (NCl-H520, ATCC-HTB-182, available from American Type Culture Collection, Rockville, Md. US) are isolated from a sample of a lung mass taken from a patient with squamous cell carcinoma of the lung (Banks-Schlegel et at Cancer Res, 45(3): 1187-1197 (1985).

H520 tumor cells are grown in RPMI 1640 medium containing 100 units/mL penicillin G sodium, 100 μg/ML streptomycin sulfate, and 25 pg/mL gentamicin. The medium is supplemented with 10% fetal bovine serum, 2 mM glutamine, and 1 mM sodium pyruvate, and buffered with 10 mM HEPES and 0.075% sodium bicarbonate. The cells are cultured in tissue culture flasks in a humidified incubator at 37° C., in an atmosphere of 5% CO₂ and 95% air. The cells are harvested for injection into 8-week old female nu/nu (nude) mice (Harlan Laboratories, Indianapolis, Ind.) by detaching the monolayers with 1× trypsin and resuspending at 5×10⁷cells/mL in phosphate-buffered saline containing 50% Matrigel.

0.2 mL of H520 tumor cell suspension (1×10⁷ cells) is injected subcutaneously in the right flank of 8-week old female nu/nu (nude) mice (Harlan Laboratories, Indianapolis, Ind.) having a body weight (BW) range of 18.1-26.9 g on Day 1 of the study. Tumors are monitored twice weekly and then daily as their mean volume approaches 120-180 mm³. Eight days after implantation, the mice are sorted into 11 groups of eight mice having individual tumor sizes of 126-196 mm³ and a group mean tumor size of 151-153 mm³, Tumor volume in mm³ is determined using the formula [(width)²×(length)]/2, where width=width of the tumor in mm and length length of the tumor in mm. Tumor weight can be estimated with the assumption that 1 mg is equivalent to 1 mrn³ of tumor volume.

The 11 groups of nude mice are treated as follows. One group of eight mice serves as Controls (C or Control) for all analyses and is administered intraperitoneally (i.p.) 50 mM sodium acetate at pH 4 (Vehicle 1) and then administered by oral gavage (p.o.) a composition comprising 10% N-methylpyrrolidone: 90% polyethylene glycol 300 (PEG 300) (Vehicle 2). All treatments with metformin (metformin hydrochloride, Glucophage®, Bristol-Myers Squibb Company) monotherapy are administered intraperitoneally (i.p,) once daily at a single dose of 192.3 mg/kg metformin until the end of the study as provided in the Results Table 2. Metformin is dissolved in 50 mM sodium acetate at pH 4 for dosing,

All treatments with the Compound A or Compound C monotherapy are administered at varying doses by oral gavage (p.o.) once daily until the end of the study as provided in the Results Table 2, Compound A is stored at −20° C. Compound C is stored at −20° C. protected from light. Stock solutions (10×) in 100% N-methylpyrrolidone (NMP) are prepared every five days, aliquotted, and stored in the dark at room temperature. On each treatment day, stock solution aliquots are diluted with polyethylene glycol (PEG300) to provide the formulated drug (Compound A or Compound C) in 10% NMP: 90% PEG300, Dosing solutions are protected from light and the formulated drug is administered within 1 hour after preparation.

For combination therapies, Compound A or Compound C is administered by oral gavage (p.o.) within 30 minutes after the intraperitoneal (i.p.) administration of metformin, except on Day 20 when Compound A or Compound C is given immediately after metformin. Compound A, Compound C and metformin are each prepared and administered as disclosed above for the monotherapy and in Results Table 2.

Paclitaxel (Natural Pharmaceuticals, Inc., Beverly, Mass., USA) is administered by bolus tail-vein injections (i.v.) once daily on alternate days for five doses. Paclitaxel is dissolved in 50% ethanol and 50% Cremophor® EL to prepare a 10× stock solution stored at room temperature. On each day of dosing, an aliquot of the pad itaxel stock solution is diluted with 5% dextrose in water to yield a dosing solution containing 5% ethanol and 5% Cremophor® EL.

In all groups, the dosing volume of 10 mL/kg (0.2 mL/20 g mouse) is scaled to the weight of each animal as determined on the day of dosing, except on weekends when the previous BW is carried forward, Acceptable toxicity for the maximum tolerated dose (MTD) is defined as a group mean BW loss of less than 15% during the test, and not more than one treatment-related mortality among ten animals. Any animal with BW losses exceeding 15% for three consecutive measurements, or with a BW loss exceeding 20% for one measurement, is designated to be euthanized.

Short-term efficacy for tumor growth inhibition in H520 cells is determined on Day 20, the day on which the Control mean tumor volume nearly attained the 1000 mm endpoint, By Day 20, no tumors had progressed to the endpoint; but 16 animals had died or been euthanized prior to Day 20. Statistical and graphical analyses was conducted by determining the difference in tumor volume between Day 1 (the start of dosing) and the endpoint day for each animal that remained on study on Day 20. Antitumor activity is expressed as % T/C (comparing the mean tumor volume change between the endpoint day and Day 1 for the treatment group to the Control). A T/C≦40% is classified as potential therapeutically active. A partial regression indicates that the tumor volume was 50% or less of its initial volume on Day 1 for three consecutive measurements during the study, and equal to or greater than 13.5 mm³ for one or more of these three measurements. A complete regression indicates that the tumor volume was less than 13.5 mm³ for three consecutive measurements during the course of the study.

Results Table 2 summarizes results for H520 tumors, for the standard 20 day experiment. The metformin monotherapy at 192.3 mg/kg did not appear to modulate tumor growth in the H520 human NSCLC xenograft model in a 20-day tumor growth inhibition assay. The paclitaxel monotherapy at 30 mg/kg produced 5% T/C and statistically significant median tumor reduction (p<0.001) as compared to Control.

The combined treatment of 32.7 mg/kg of COMPOUND C plus metformin resulted in 62% T/C but was not statistically evaluable due to two deaths. The combined treatment of 54.4 mg/kg of COMPOUND C plus metformin resulted in 125% T/C but was not statistically evaluable due to five deaths.

The combined treatment of 41.1 mg/kg of COMPOUND A plus metformin resulted in 60% T/C, which shows an improved growth inhibition that is not statistically significant as compared to Control. The combined treatment of 68.5 mg/kg of COMPOUND A plus metformin resulted in 36% T/C which is an improvement over the corresponding COMPOUND A monotherapy. The results for the 68.5 mg/kg of COMPOUND A combination therapy and monotherapy were not statistically evaluable due to three deaths.

The combination of COMPOUND A plus metformin inhibits the growth of human NSCLC cell lines. It is demonstrated that the combination treatment of COMPOUND A plus metformin improved inhibition of growth of human NSCLC cell lines as compared to Control and the COMPOUND A monotherapy. Metformin may potentially increase the toxicity of COMPOUND A and/or COMPOUND C in this H520 xenograft model.

TABLE 2
Antitumor effect of PI3K inhibitors and metformin, alone and in
combination, on H520 non-small cell lung tumors in nude mice

| | | | Mean Tumor | | | | |
|---|---|---|---|---|---|---|---|
| | Dose | Route, | Vol. Change | | | Mean Body | Dead/ |
| Compound | (mg/kg) | Schedule | (mm³) | Regression | T/C | Weight Change | Total |
| Vehicle 1/ | — | i.p., 1x daily | 996 | None | — | — | 0/8 |
| Vehicle 2 | | p.o., 1x daily | | | | | |
| Metformin | 192.3 | i.p., 1x daily | 1003 | None | 101% (ns^{†}) | — | 0/8 |
| Compound C | 32.7 | p.o., 1x daily | 542 | None | 54% (ns^{†}) | — | 0/8 |
| Compound C | 54.4 | p.o., 1x daily | 475 | None | 48% (ne) | −107%, Day 7 | 4/8 |
| | | until day 7^{‡} | | | | | |
| Compound A | 41.1 | p.o., 1x daily | 598 | None | 60% (ns^{†}) | — | 0/8 |
| Compound A | 68.5 | p.o., 1x daily | 466 | None | 47% (ns^{†}) | −1.3%, Day 7 | 1/8 |
| Metformin | 192.3 | i.p., 1x daily | 618 | None | 62% (ne) | — | 2/8 |
| Compound C | 32.7 | p.o., 1x daily | | | | | |
| Metformin | 192.3 | i.p., 1x daily | 1245 | None | 125% (ne) | −11.8%, Day 7 | 5/8 |
| | | until day 5^{‡} | | | | | |
| Compound C | 54.4 | p.o., 1x daily | | | | | |
| | | until day 5^{‡} | | | | | |
| Metformin | 192.3 | i.p, 1x daily | 579 | None | 58% (ne) | −2.3%, Day 7 | 2/8 |
| Compound A | 41.1 | p.o., 1x daily | | | | | |
| Metformin | 192.3 | i.p., 1x daily | 358 | None | 36% (ne) | −4.4%, Day 7 | 2/8 |
| | | until day 8^{‡} | | | | | |
| Compound A | 68.5 | p.o., 1x daily | | | | | |
| | | until day 8^{‡} | | | | | |
| Paclitaxel | 30 | i.v., 1x on | 53 | None | 5% | — | 0/8 |
| | | alternate days | | | (p < 0.001)^{†} | | |
| | | for 5 doses | | | | | |

^{†}Statistical Significance (ANOVA with post-hoc Dunnett's multiple comparison test vs. control, except for the group treated with paclitaxel; Kruskal-Wallis and post-hoc Dunn's multiple comparison test vs. control, including the group treated with paclitaxel): ne = not evaluable; ns = not significant
^{‡}Treatment stopped at specified day due to toxicity
T/C = 100 × (ΔT/ΔC) = percent change between Day 1 and D 20 in the mean tumor volume of a treated group (ΔT) compared with change in Control (ΔC).
Mean BW Change = lowest group mean body weight, as change from Day 1 up to Day 20, “—” indicates no decrease in mean body weight was observed.

### Example 4

### Combination Effect of PI3K Inhibitors and Metformin on H460 Non Small Cell Lung Tumors in Nude Mice

H460 human non-small cell lung cancer (NSCLC) cells are treated with, different doses of 2-Methyl-2-[4-(3-methyl-2-oxo-8-quinolln-3-yl-2,3-dihydro-imidazo[4, 5-c]quinolin-1-yl)-phenyl]-propionitrile monotosylate salt (also known as COMPOUND A) or 5,-(2,6-di-rnorpholin-4-yl-pyrimidin-4-yl)-4-trifluoromethyl-pyridin-2-ylamine hydrochloride salt (also known as COMPOUND C), a single dose of metformin or both agents in combination, The H460 human NSCLC cells (NCl-H460. ATCC-HTB-177, available from American Type Culture Collection, FRockville, Md. US) are derived from the pleural fluid of a patient with large cell cancer of the lung.

H460 tumor cells are grown in RPMI 1640 medium containing 100 units/mL penicillin G sodium, 100 μg/mL streptomycin sulfate, and 25 μg/mL gentamicin. The medium is supplemented with 10% fetal bovine serum and 2 mM glutamine. The cells are cultured in tissue culture flasks in a humidified incubator at 37° C., in an atmosphere of 5% CO₂ and 95% air. The cells are harvested for injection into 9-week old female nu/nu (nude) mice (Harlan Laboratories, Indianapolis, Ind.) by detaching the monolayers with 2× trypsin and resuspending at 5×10⁷ cells/mL in phosphate-buffered saline.

0.2 mL of H460 tumor cell suspension (1×10⁷ cells) is injected subcutaneously in the right flank of 9-week old female nu/nu (nude) mice (Harlan Laboratories, Indianapolis, Ind.) having a body weight (BW) range of 18.1-26.9 g on Day 1 of the study. Tumors are monitored twice weekly and then daily as their mean volume approaches 100-150 mm³. Ten days after implantation, the mice are sorted into 11 groups of eight mice having individual tumor sizes of 75-196 mm³ and a group mean tumor size of 119-122 mm³. Tumor volume in mm³ is determined using the formula [(width)²×(length)]/2, where width width of the tumor in mm and length length of the tumor in mm. Tumor weight can be estimated with the assumption that 1 mg is equivalent to 1 mm³ of tumor volume.

The 11 groups of nude mice are treated as follows. One group of eight mice serves as Controls (C or Control) for all analyses and is administered intraperitoneally (i.p.) 50 mM sodium acetate at pH 4 (Vehicle 1) and then administered by oral gavage (p.o.) a composition comprising 10% N-methylpyrrolidone 90% polyethylene glycol 300 (PEG300) (Vehicle 2). All treatments with metformin (metformin hydrochloride, Glucophage®, Bristol-Myers Squibb Company) monotherapy are administered intrapeftneally (i.p.) once daily at a single dose of 192.3 mg/kg metformin until the end of the study as provided in the Results Table 3. Metformin is dissolved in 50 mM sodium acetate at pH 4 for dosing.

All treatments with the Compound A or Compound C monotherapy are administered at varying doses by oral gavage (p.o.) once daily until the end of the study as provided in the Results Table 3. Compound A is stored at −20° C. Compound C is stored at −20° C. protected from light. Stock solutions (10×) in 100% N-rnethylpyrrolidone (NMP) are prepared every five days, aliquotted, and stored in the dark at room temperature. On each treatment day, stock solution aliquots are diluted with polyethylene glycol (PEG300) to provide the formulated drug (Compound A or Compound C) in 10% NMP: 90% PEG300. Dosing solutions are protected from light, and the formulated drug is administered within 1 hour after preparation.

For combination therapies, Compound A or Compound C are administered by oral gavage (p.o.) within 30 minutes after the intraperitoneal (i.p.) administration of metformin, except on Day 20 when Compound A or Compound C is given immediately after metformin. Compound A, Compound C and metformin are each prepared and administered as disclosed above for the monotherapy and in Results Table 3.

Paclitaxel (Natural Pharmaceuticals, Inc., Beverly, Mass., USA) is administered by bolus tail-vein injections (i.v.) once daily on alternate days for five doses. Paclitaxel is dissolved in 50% ethanol and 50% Crernophor® EL to prepare a 10× stock solution stored at room temperature. On each day of dosing, an aliquot of the paclitaxel stock solution is diluted with 5% dextrose in water to yield a dosing solution containing 5% ethanol and 5% Cremophor® EL.

In all groups, the dosing volume of 10 mL/kg (0.2 mL/20 g mouse) is scaled to the weight of each animal as determined on the day of dosing, except on weekends when the previous BW is carried fontvard. Acceptable toxicity for the maximum tolerated dose (MTD) is defined as a group mean BW loss of less than 15% during the test, and not more than one treatment-related mortality among ten animals. Any animal with BW losses exceeding 15% for three consecutive measurements, or with a BW loss exceeding 20% for one measurement, is designated to be euthanized.

Short-term efficacy for tumor growth inhibition in H460 cells is determined on Day 12, the day on which the Control mean tumor volume nearly attained the 1000 mm³ endpoint. Statistical and graphical analyses was conducted by determining the difference in tumor volume between Day 1 (the start of dosing) and the endpoint day for each animal that remained on study on Day 12. Antitumor activity is expressed as % T/C (comparing the mean tumor volume change between the endpoint day and Day 1 for the treatment group to the Control). A T/C≦40% is classified as potential therapeutically active. A partial regression indicates that the tumor volume was 50% or less of its initial volume on Day 1 for three consecutive measurements during the study, and equal to or greater than 13.5 mm³ for one or more of these three measurements. A complete regression indicates that the tumor volume was less than 13.5 mm³ for three consecutive measurements during the course of the study.

Results Table 3 summarizes results for H460 tumors, for the standard 12 day experiment. The metformin monotherapy at 192.3 mg/kg did not appear to impact tumor growth in the H460 human NSCLC xenograft model in a 12-day tumor growth inhibition assay. The response to paclitaxel is consistent with prior results reported in this xenograft model.

The combination treatment of 41.1 mg/kg of COMPOUND A plus metformin results in statistically significant improved inhibition of tumor growth at 19% T/C as compared to the Control (p<0.05 when analyzed with Kruskal-Wallis and post-hoc Dunn's multiple comparison test, p<0.01 when analyzed with ANOVA with post-hoc Dunnett's multiple comparison test). The combination treatment of 41.1 mg/kg of COMPOUND A plus metformin further results in statistically significant improved inhibition of tumor growth at 19% as compared to the metformin monotherapy (p<0.01 when analyzed with ANOVA with post-hoc Dunnett's multiple comparison test) and non-statistically significant improvement over the COMPOUND A monotherapy. The combination treatment of 68.5 mgfkg of COMPOUND A plus metformin results in improved inhibition of tumor growth at 31% T/C as compared to Control. The combination treatment of 68.5 mg/kg of COMPOUND A plus metformin further results in statistically significant improved inhibition of tumor growth at 31% as compared to the metformin monotherapy (p<0.01 when analyzed with ANOVA with post-hoc Dunnett's multiple comparison test) and no improvement over the COMPOUND A monotherapy.

The combination treatment of 32.7 mg/kg of COMPOUND C and metformin did not improve inhibition of tumor growth at 59% T/C as compared to the corresponding COMPOUND C monotherapy. Combination therapy with 54.4 COMPOUND C and metformin was terminated early due to toxicity.

The combination of COMPOUND A plus metformin inhibits the growth of human NSCLC cell lines. It is demonstrated that the combination treatment of COMPOUND A plus metformin improved inhibition of growth of human NSCLC cell lines as compared to Control, the metformin monotherapy and the COMPOUND A monotherapy, However, metformin does not appear to enhance efficacy or tolerability in combination with COMPOUND C in this H460 xenograft model.

TABLE 3
Antitumor effect of PI3K inhibitors and metformin, alone and in
combination, on H460 non-small cell lung tumors in nude mice

| | | | Mean Tumor | | | | |
|---|---|---|---|---|---|---|---|
| | Dose | Route, | Vol. Change | | T/C | Mean Body | Dead/ |
| Compound | (mg/kg) | Schedule | (mm³) | Regression | (SS1, SS2) | Weight Change | Total |
| Vehicle 1/ | — | i.p., 1x daily | 1502 | None | — | — | 0/8 |
| Vehicle 2 | | p.o., 1x daily | | | | | |
| Metformin | 192.3 | i.p., 1x daily | 1516 | None | 101% (ns, | — | 0/8 |
| | | | | | ns) | | |
| Compound C | 32.7 | p.o., 1x daily | 880 | None | 59% (ns, | — | 0/8 |
| | | | | | ns) | | |
| Compound C | 54.4 | p.o., 1x daily | 110 | None | 7% (ne, | −10%, Day 12 | 4/8 |
| | | until day 10^{‡} | | | ne) | | |
| Compound A | 41.1 | p.o., 1x daily | 809 | None | 54% (ns, | 00 | 0/8 |
| | | | | | ns) | | |
| Compound A | 68.5 | p.o., 1x daily | 448 | None | 30% (ns, | — | 0/8 |
| | | | | | p < 0.05) | | |
| Metformin | 192.3 | i.p., 1x daily | 886 | None | 59% (ns, | — | 0/8 |
| Compound C | 32.7 | p.o., 1x daily | | | ns) | | |
| Metformin | 192.3 | i.p., 1x daily | 68 | None | 5% (ne, | −15.1%, Day 12 | 4/8 |
| | | until day 10^{‡} | | | ne) | | |
| Compound C | 54.4 | p.o., 1x daily | | | | | |
| | | until day 10^{‡} | | | | | |
| Metformin | 192.3 | i.p., 1x daily | 279 | None | 19% | — | 0/8 |
| Compound A | 41.1 | p.o., 1x daily | | | (p < 0.05, | | |
| | | | | | p < 0.01) | | |
| Metformin | 192.3 | i.p., 1x daily | 465 | None | 31% (ns, | — | 0/8 |
| Compound A | 68.5 | p.o., 1x daily | | | p < 0.05) | | |
| Paclitaxel | 30 | i.v., 1x on | 227 | None | 15% | −9.7, Day 12 | 0/8 |
| | | alternate days | | | (p < 0,05, —) | | |
| | | for 5 doses | | | | | |

^{‡}Treatment stopped at Day 10 due to toxicity
T/C = 100 × (ΔT/ΔC) = percent change between Day 1 and D 20 in the mean tumor volume of a treated group (ΔT) compared with change in Control (ΔC).
SS1 = Statistical Significance (Kruskal-Wallis and post-hoc Dunn's multiple comparison test) as compared to Control: ne = not evaluable; ns = not significant
SS2 = Statistical Significance (ANOVA with post-hoc Dunnett’s multiple comparison test, excluding the group treated with Paclitaxel) as compared to Control: ne = not evaluable; ns = not significant
Mean BW Change = lowest group mean body weight, as change from Day 1 up to Day 12, “—” indicates no decrease in mean body weight was observed.

1-18. (canceled)

19. A combination which comprises (a) a phosphoinositide 3-kinase inhibitor selected from the group consisting of a compound of formula I 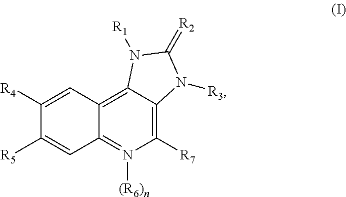
wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl;
pyrazolyl; and triazolyl;
R₂ is O or S;
R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy
R₅ is hydrogen or halogen;
n is 0 or 1;
R₆ is oxido;
with the proviso that if n=1, the N-atom bearing the radical R₆ has a positive charge;
R₇ is hydrogen or amino;
or a tautomer thereof, or a pharmaceutically acceptable salt, or a hydrate or solvate thereof, and
a compound of formula II 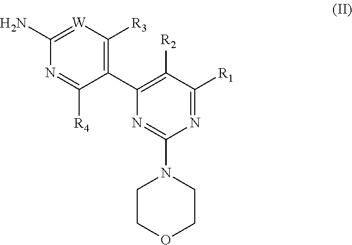
wherein,
W is CR_{w} or N, wherein R_{w} is selected from the group consisting of
(1) hydrogen,
(2) cyano,
(3) halogen,
(4) methyl,
(5) trifiuoromethyl,
(6) sulfonamido;
R₁ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyt,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NRᵢₐSO₂R_{1b},
(18) —OCOR₁ₐ,
(19) —OR₁ₐ,
(20) —SR₁ₐ,
(21) —SOR₁ₐ,
(22) —SO₂R₁ₐ, and
(23) —SO₂NR₁ₐR_{1b},
wherein R₁ₐ, and R_{1b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
R₂ is selected from the group consisting
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b},
wherein R₂ₐ, and R_{2b} are independently selected from the group consisting of
(a) hydrogen, and
(b) substituted or unsubstituted alkyL
R3 is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(13) —NR₃ₐR_{3b},
(14) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, and
(20) —SO₂NR₃ₐR_{3b},
wherein R₃ₐ, and R_{3b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and
R₄ is selected from the group consisting of
(1) hydrogen, and
(2) halogen.
or a stereoisomer, tautomer, or pharmaceutically acceptable salt thereof
and (b) a insulin sensitivity enhancer compound, wherein the active ingredients are present in each case in free form or in the form of a pharmaceutically acceptable salt or any hydrate thereof, and optionally at least one pharmaceutically acceptable carrier; for simultaneous, separate or sequential use.

20. A combination according to claim 19, wherein the phosphoinositide 3-kinase inhibitor is 2-methyl-2-[4-(3-methyl-2-oxo-8-quinolin-3-yl-2,3-dihydro-imidazo[4,5-c]quinolin-1-yl)-phenyl]-propionitrile and its monotosylate salt, 8-(6-methoxy-pyridin-3-yl)-3-methyl-1-(4-piperazin-1-yl-3-trifluoromethyl-phenyl)-1,3-dihydro-imidazo[4,5-c]quinolin-2-one or 5-(2,6-di-morpholin-4-yl-pyrimidin-4-yl)-4-trifluoromethyl-pyridin-2-ylamine or a pharmaceutically acceptable salt thereof.

21. A pharmaceutical composition comprising a quantity of a combination according to claim 19 and at least one pharmaceutically acceptable carrier, which is jointly therapeutically effective against a proliferative disease.

22. A pharmaceutical composition comprising a combination according to claim 19 wherein the insulin sensitivity enhancer compound is is a biguanide or glitazone.

23. A combination according to claim 19 for use in the treatment of a proliferative disease and/or overcoming the potential increase in blood glucose caused by inhibition of the PI3K/Akt pathway.

24. A method of treating a patient suffering from a proliferative disease comprising administering an effective amount of a phosphoinositide 3-kinase inhibitor compound of claims 19 and an insulin sensitivity enhancer compound, wherein the active ingredients are present in each case in free form or in the form of a pharmaceutically acceptable salt or any hydrate thereof, and optionally at least one pharmaceutically acceptable carrier, for simultaneous, separate or sequential use.

25. The method according to claim 24 wherein the proliferative disease is a solid tumor disease.

26. The method according to claim 24 wherein the proliferative disease is lung tumors carrying a loss-of-function mutation of LKB1.

27. The method according to claim 24 wherein the proliferative disease is Breast Cancer Ovarian Cancer, Colon Cancer, Pancreas Cancer, Melanoma, Head and Neck, Brain Cancer, Endometrial Cancer, Cancers in patients with Peutz Jeghers Syndrome.

28. The method according to claim 24 wherein the patient is overcoming the potential increase in blood glucose caused by inhibition of the PI3K/Akt pathway.

29. The method according to claim 24, wherein the phosphoinositide 3-kinase inhibitor compound is selected from 2-methyl-2-[4-(3-methyl-2-oxo-8-quinolin-3-yl-2, 3-dihydro-imidazo[4,5-c]quinolin-1-yl)-phenyl]-propionitrile and its monotosylate salt, 8-(6-methoxy-pyridin-3-yl)-3-methyl-1-(4-piperazin-1-yl-3-trifluoromethyl-phenyl)-1, 3-dihydro-imidazo[4,5-c]quinolin-2-one or 5-(2,6-di-morpholin-4-yl-pyrimidin-4-yl)-4-trifluorornethyl-pyridin-2-ylamine.

30. The method according to claim 24, wherein the insulin sensitivity enhancer compound is a biguanide or glitazone.

31. A combined preparation, which comprises (a) one or more unit dosage forms of phosphoinositide-3 kinase inhibitor according of claim 19 and (b) one or more unit dosage forms of a biguanide or glitazone insulin sensitivity enhancer compound.
